(19) United States
(12) Patent Application Publication
(10) Pub. No.: US 2006/0217637 A1
(43) Pub. Date: Sep. 28, 2006

(54) Anastomotic leak testing apparatus

(76) Inventors: Arnold R. Leiboff, Stony Brook, NY (US); Joseph Zipper, Miller Place, NY (US)

Correspondence Address: BRIAN ROFFE, ESQ, 11 SUNRISE PLAZA, SUITE 303, VALLEY STREAM, NY, 11580-6170, US

(21) Appl. No.: 11/444,783

(22) Filed: Jun. 1, 2006

Related U.S. Application Data
(63) Continuation-in-part of application No. 10/664,366, filed on Sep. 17, 2003.

(51) Int. Cl. A61B 5/103

(52) U.S. Cl. 600/593

(57) ABSTRACT

Anastomotic leak tester for testing for leaks in a gastrointestinal anastomosis in a patient including an air introduction system adapted to be inserted into the patient's gastrointestinal tract through a natural body orifice of the patient and enabling air flow therethrough into the gastrointestinal tract and a regulating mechanism arranged in connection with the air introduction system for regulating air pressure in the patient's gastrointestinal tract. The regulating mechanism may include a pressure relief valve having an inlet communicating with an interior space of the air introduction system communicating with the patient's gastrointestinal tract and which allows air to be released from the patient's gastrointestinal tract when air pressure in the patient's gastrointestinal tract exceeds a predetermined pressure. The pressure relief valve may generate an audible indication when air pressure in the patient's gastrointestinal tract exceeds the predetermined pressure.

### CROSS REFERENCE TO RELATED APPLICATION

This application is a continuation-in-part of U.S. patent application Ser. No. 10/664,366 filed Sep. 17, 2003, which is incorporated by reference herein.

### FIELD OF THE INVENTION

The present invention relates to anastomotic leak testing apparatus including an inflation pump and an air introduction device for introducing air into the bowel of a patient during a surgical operation to remove a portion of the rectum and/or distal colon in order to evaluate the integrity of an anastomosis. The present invention also relates to methods for testing for anastomotic leakage and for visualization of a completed anastomosis.

The present invention also relates to anastomotic leak testing apparatus and methods for use in testing a gastrointestinal anastomosis for leaks, which regulate air pressure in the anastomosed portion of the gastrointestinal tract by releasing air when the pressure exceeds a predetermined pressure to prevent overdistending the anastomosed portion of the gastrointestinal tract and to maintain intraluminal pressure within a predetermined range provided there is not an anastomotic leak.

The present invention also relates to anastomotic testing apparatus and methods that provide an audible or visual indication when the pressure in the anastomosed portion of the gastrointestinal tract reaches or exceeds a predetermined pressure to notify the surgeon that the anastomosis is sufficiently distended to detect an anastomotic leak.

### BACKGROUND OF THE INVENTION

Surgical operations to remove the distal colon and part of the rectum are common for illnesses such as neoplasia, diverticular disease and inflammatory bowel disease. When the continuity of the gastrointestinal tract is to be preserved, the bowel must be reconnected by means of sutures, staples or a compression device. This connection is called an anastomosis.

If there is a defect in the anastomosis, gastrointestinal content can leak out of the bowel and contaminate the normally sterile peritoneal cavity, causing peritonitis. Peritonitis (infection of the peritoneal cavity) can be lethal, and therefore measures must be taken during surgery to ensure that leaks in the anastomosis are not present. One such measure is to fill the pelvic portion of the abdominal cavity with saline or water to a level which submerses a completed colorectal anastomosis, then insufflate the bowel with air while occluding the bowel above the anastomosis. As the bowel distends, the pool of saline is observed for air bubbles which, if present, signal a defect in the anastomosis, i.e., a leak, which must then be repaired.

One method to insufflate the bowel with air is to use a syringe, e.g., a bulb syringe or catheter-tipped Toomey syringe, to inject air through the anus into the bowel. After the first injection, the syringe is removed, allowed to fill with air, and then re-inserted into the anus to inject more air. This process is repeated until the surgeon is satisfied with the degree of bowel distension.

Another method currently used to insufflate the bowel is to insert a catheter through the anus into the bowel and through the catheter inject air into the bowel with a large syringe. The catheter may or may not have an inflatable retention cuff near its tip. A Foley catheter is an example of a useful catheter with an inflatable cuff which is readily available in an operating room. Air is injected into the bowel through the catheter, the catheter is then clamped or crimped, the syringe is detached, refilled with air, reattached to the catheter, and more air is injected. This cycle is continued until the bowel is sufficiently distended with air. This process utilizes both a catheter and a syringe and is somewhat cumbersome.

A more frequently used device to insufflate air into the rectum is a type of endoscope called a proctoscope (or sigmoidoscope), i.e., a rigid tubular instrument designed for looking into the rectum. The proctoscope has an inflation pump attached to it. With the lens gate of the proctoscope closed, air is introduced into the rectum by compressing the inflation pump.

Use of a proctoscope for rectal insufflation to test for anastomotic leaks has advantages and disadvantages. The fact that proctoscopes are items usually stocked in an operating room is an advantage. Thus, an additional surgical instrument does not need be purchased or stocked in an operating room in order to test for anastomotic leaks. Moreover, it is advantageous that it is possible to visually inspect the anastomosis from inside the bowel using the proctoscope. This helps identify potentially harmful bleeding, which can then be stopped by suture ligation.

On the other hand, a significant disadvantage of the use of a proctoscope results from the fact that a reusable proctoscope has to be cleaned, repackaged and sterilized after every use. Much of the cost savings gained by using an instrument already in stock and accessible may be lost by the extra labor involved in cleaning, repackaging and sterilizing the proctoscope after surgery. Disposable proctoscopes (rigid sigmoidoscopes) are available and may be used, but they must be used with reusable components, such as the light source handle and inflation pump, which must be maintained in operating condition and cleaned, repackaged and sterilized after every use.

An additional disadvantage of using a proctoscope to insufflate the bowel is that the insufflated air tends to leak out through the anus around the proctoscope, which typically has an outer diameter of about 0.75 inches. Extra effort may therefore be required to distend the bowel sufficiently to effectively test the anastomosis. It is also possible to over-distend the bowel with air insufflation and actually disrupt the anastomosis. It is not possible to accurately gauge the level of air pressure in the bowel because the proctoscope does not provide any mechanism to measure the air pressure in the bowel, and thus the air pressure can only be grossly evaluated by visual inspection of the distended bowel above the pool of saline, or by feeling the bowel.

Yet another drawback of the use of a proctoscope is that although the proctoscope can be used to visually inspect the stapled anastomosis inside the bowel, the view obtained via a standard proctoscope is relatively poor, is not sufficiently keen to allow the surgeon to spot anastomotic defects and usually requires the surgeon to crouch in an awkward position and spend some time to achieve a satisfactory inspection.

Furthermore, many surgeons use a proctoscope to examine a tumor or cleanse the rectum at the start of a colorectal operation, before opening the abdomen. The proctoscope must then either be kept in the operating room in a dirty state, or cleansed in a nearby utility room by a nurse, if the surgeon wants to use the same proctoscope later in the case to test an anastomosis. This not only clutters the operating room, but can potentially contaminate the operating room, and in any event, necessitates extra labor by a circulating nurse. If the proctoscope that was used earlier to view a tumor or cleanse the rectum is re-used without being cleaned or resterilized, then the bowel may be insufflated with fecal debris, airborne bacteria or even particles of cancerous tumor that had contaminated the proctoscope or the inflation pump or tubing. Such fecal debris, airborne bacteria or tumor particles could escape the bowel through an anastomotic leak and contribute to the development of postoperative peritonitis, wound infection or cancer recurrence. A disadvantage of all the methods described above is that the surgeon has no way to accurately determine the amount of pressure within the bowel. The surgeon may not know whether he is distending the bowel sufficiently to detect a leak, or he may not be aware that he is over-distending the bowel and thereby possibly causing injury to the newly created anastomosis, such injury could lead to post-operative failure of the anastomosis. Gilbert studied distending pressures used to test for anastomotic leaks when testing is performed with fluid, and recommended distending pressures between 25 and 30 cm water. (Gilbert J M, Trapnell J E. Intraoperative testing of the integrity of left-sided colorectal anastomoses: a technique of value to the surgeon in training, Ann R Coll Surg Engl. 1988, 70:158-160 and Wheeler J M D, Gilbert J M Ann R Coll Engl 1999; 81:105-108). However, his method using a water manometer of the type used for measuring central venous pressure was felt to be too cumbersome for routine use in the operating room.

Thus, there are significant drawbacks to the use of all the afore-mentioned techniques to insufflate air into the bowel and to inspect an anastomosis and it would be desirable to provide alternative means to insufflate the bowel and inspect the anastomosis which avoid these drawbacks.

### OBJECTS AND SUMMARY OF THE INVENTION

It is an object of the present invention to provide simple, inexpensive and disposable devices to be used during surgery for insufflation of the bowel with air to test for gastrointestinal anastomotic leakage.

It is another object of the present invention to provide new methods for testing for anastomotic leaks using novel anastomotic leak testers.

Another object of the present invention is to provide an inexpensive anastomotic leak tester that can lead to cost savings and more efficient use of operating room time and personnel.

Another object of the present invention is to provide a new anastomotic leak tester which provides a signal when there is sufficient air pressure in the bowel.

Another object of the present invention is to provide a new anastomotic leak tester that maintains pressure in the anastomosed portion of the gastrointestinal tract at an optimal pressure to test for anastomotic leak (approximately 25 to 30 cm water).

Another object of the present invention is to provide a new anastomotic leak tester that prevents the surgeon from over-distending the anastomosed portion of the gastrointestinal tract during leak testing and thereby prevents injury to the anastomosis.

Yet another object of this invention is to provide an anastomotic leak tester to test for leaks in anastomoses between the small bowel or colon and the colon, rectum or anus.

It is still another object of the present invention to provide an anastomotic leak tester which can be inserted through the anus to test anastomoses to the colon, rectum or anus.

It is still another object of the present invention to provide an anastomotic leak tester which is easier to use than a proctoscope for the purpose of testing an anastomosis for leaks.

It is yet another object of the present invention to provide an air introduction device for testing for anastomotic leaks which is safer to use than a standard proctoscope.

Still another object of the present invention is to provide a new and improved anastomotic leak tester that enables improved visualization of the anastomosis from within the bowel.

Still another object of the present invention is to provide an adapter for a sigmoidoscope that provides a signal when there is sufficient air pressure in the bowel.

Still another object of the present invention is to provide an adapter for a sigmoidoscope that maintains pressure in the bowel at an optimal pressure to test for anastomotic leak (approximately 25 to 30 cm water).

Still another object of the present invention is to provide an adapter for a sigmoidoscope that prevents the surgeon from over-distending the bowel during leak testing and thereby prevents injury to the anastomosis.

Yet another object of this invention is to provide an anastomotic leak tester to test for leaks in anastomoses between the stomach and small bowel.

In order to achieve these objects and others, an anastomotic leak tester for testing for leaks in a colorectal anastomosis in a patient in accordance with the invention includes an inflation pump having a bulbous compressible portion and a mechanism for providing a uni-directional flow of air through the compressible portion upon intermittent compressing thereof, and an air introduction device comprising a body having an interior space, a proximal portion having an opening communicating with the interior space and adapted to be inserted into an anus of a patient and a distal portion arranged to engage with the inflation pump. Upon compression of the compressible portion of the inflation pump, air flows through the inflation pump into the interior space of the body and through the opening into the patient. The inflation pump may be a commercially available device.

In some embodiments, the inflation pump is removably attached to the air introduction device, i.e., two separate components are provided, whereas in other embodiments, the inflation pump is formed integral with the air introduction device. In the former case, a connection mechanism is provided to operatively connect the inflation pump to the distal portion of the body of the air introduction device, e.g., to an axial end of the body. One connector mechanism is a connector arranged either on the inflation pump to mate with a connector portion on the distal portion of the body or on the distal portion of the body and arranged to mate with a connector portion at a proximal end of the inflation pump.

The body of the air introduction device can have different forms. For example, it may be elongate, flexible, tubular, opaque, translucent or transparent. One specific form of the body includes a proximal portion constructed to cause the anus to constrict around it and thereby form a seal against the anal wall and an expanded portion having a larger size than the proximal portion and interposed between the proximal portion and the distal portion. The expanded portion occludes the anus by engaging with the anal opening to limit insertion of the proximal portion into the anus and seal the elastomeric body against the anal opening.

In one embodiment, the distal portion includes only a single arm, i.e., a single tubular segment defining a lumen which receives the inflation pump.

In another embodiment, the distal portion has two arms, one arm defining a lumen which receives a connector of the inflation pump and the other arm defining a lumen through which a visualization device or endoscope such as a laparoscope may be inserted. Thus, one arm serves as an inflation arm and the other serves as an endoscopic port.

Instead of or in addition to an arm providing an endoscopic port, an arm can be provided to retain a pressure relief valve for releasing air when a specific air pressure in the bowel is reached. This prevents over-inflation of the bowel. A signal mechanism can be associated with the valve to provide a signal when air is released via the valve, e.g., an extension coupled to the arm and including vibrating flaps which vibrate and produce an audible sound when air is released via the valve. Alternatively, the functions of a pressure relief valve and signal mechanism can be incorporated into a single element which opens and vibrates only when a critical air pressure within the bowel is reached.

Another way to consider the air introduction device in accordance with the invention would be as a unitary body defining an interior space and having an insertion and sealing mechanism for enabling insertion of a part of the body into an anus of a person such that the anus constricts around the part and thereby seals the body against anal walls, an insertion-limiting mechanism for limiting insertion of the part of the body into the anus and occluding the opening of the anus, and a coupling mechanism for enabling coupling of the body to an inflation pump such that air is directable from the inflation pump through the coupling mechanism into the interior space in the elastomeric body. The insertion and sealing mechanism may be the proximal portion of the body discussed above. The insertion-limiting and anal occluding mechanism may be the expanded portion of the body discussed above. The coupling mechanism may be a lumen arranged on a distal portion of the body and adapted to receive a connector of the inflation pump.

An exemplifying method for anastomotic leak testing of a colorectal anastomosis in accordance with the invention includes the steps of providing an air introduction device comprising an elongate body having an interior space, a proximal portion having an opening communicating with the interior space and a distal portion, coupling an inflation pump to the distal portion of the body, inserting the proximal portion of the body into the anus of a patient to cause the proximal portion to dilate the anus whereby the anus constricts around the proximal portion, filling the pelvis of the patient with fluid, occluding the bowel proximal to the anastomosis, compressing the inflation pump one or more times to cause air to flow through into the interior space of the body and out of the opening into the bowel to thereby distend the bowel of the patient with air, and once the bowel is sufficiently distended, checking for anastomotic leaks based on the presence of air bubbles in the pelvic fluid. If the inflation pump is detachably coupled to the distal portion of the body, it can be detached from the body after checking for anastomotic leaks while the proximal portion of the body remains inserted in the anus thereby allowing air from the bowel to escape from the bowel and the bowel to deflate. The body is then removed from the anus.

The body may include a second arm having a lumen through which a visualization device or endoscope such as a laparoscope may be inserted. Initially, the end of the second arm may be closed but after the initial anastomotic leak check described above is performed, the body is removed from the anus and the closed end of the second arm is opened. An endoscope may then be inserted into the lumen so that the tip of the endoscope sits within a space of the elastomeric body. The proximal portion of the body is re-inserted into the anus and the inflation pump is again compressed to distend the bowel of the patient with air. The anastomosis is then visually inspected by advancing the tip of the endoscope from the space in the elastomeric body into the distended bowel until the anastomosis is viewable through the endoscope or on a video display to thereby enable the integrity of the anastomosis to be ascertained and to check for bleeding at the anastomosis. The endoscope is removed from the elastomeric body when the visual inspection is complete while the body is engaged with the anus and then the bowel is deflated and the body removed out of engagement with the anus.

A colorectal anastomotic leak tester in accordance with the invention, and method for using the same, has several advantages over the prior art, notably the use of a standard proctoscope for the purpose of insufflating the bowel to test for anastomotic leakage. An anastomotic leak tester in accordance with the invention is easier to use than a proctoscope because it is more compact and requires only one hand to operate, whereas a proctoscope is relatively unwieldy and requires two hands to use, one to position the proctoscope and the second to repetitively compress the inflation pump. The device in accordance with the invention is also easier to use than a proctoscope because it prevents air from escaping from the bowel around the device, resulting in fewer inflation pump compressions to achieve adequate bowel distension.

An anastomotic leak tester in accordance with the invention is also safer to use than a standard proctoscope because the device is sterile until used to insufflate the bowel and therefore no potentially hazardous debris collects in the device nor is there a possibility of debris originating from the device contaminating the peritoneal cavity (as is the case when a contaminated proctoscope is used).

The anastomotic leak tester is also designed to be an inexpensive alternative to a proctoscope and to be easier to open, assemble, use and dispose of than it would be to open, assemble, use, transport, cleanse, re-package and sterilize a proctoscope.

One embodiment of the anastomotic leak tester prevents the surgeon from inflating the bowel above a desired pressure, so that the anastomosis is not damaged by excessive pressure, by providing a pressure relief valve or other pressure relief mechanism. This embodiment also maintains the bowel intraluminal pressure within a range desirable for anastomotic leak testing (e.g., 25 to 30 cm water).

Another embodiment provides a signal when a desired air pressure within the bowel is reached. The signal may be a visual signal, an audible signal, both a visual and audible signal or any other type of signal which is indicative of the air pressure in the bowel being the desired air pressure.

Another embodiment of the anastomotic leak tester is designed for use in conjunction with a laparoscope or other image-obtaining device that can visualize the anastomosis from within the bowel to a degree of clarity which far surpasses the view that one can achieve with a rigid proctoscope.

Yet another embodiment of an anastomotic leak tester in accordance with the invention is useful for testing anastomoses to the stomach or esophagus. This anastomotic leak tester includes a long tube capable of being inserted through the nose or mouth into the stomach. This insertion tube is at least a dual lumen tube with a primary lumen having at least one hole near its forward tip. At its rearward end, this primary lumen branches into an inflation arm that accommodates an inflation pump and an arm which accommodates a pressure relief valve. The secondary lumen has a hole rearward of the forward tip of the tube, rearward of the holes in the primary lumen, through which the secondary lumen communicates with the interior of an inflation cuff that surrounds the tube. At the rearward end of the secondary lumen, a valve is present to which a syringe is adaptable to inject air into the secondary lumen to inflate the cuff, and which prevents air from leaking out of the inflated cuff unless withdrawn by means of a syringe. In use, the forward end of the tube is inserted through the nose or mouth, through the pharynx and esophagus, into the stomach. The inflation cuff is inflated and the tube is withdrawn so that the cuff abuts against the stomach wall near the gastroesophageal junction. The inflated cuff is too large to be withdrawn into the esophagus, and provides a seal at the gastroesophageal junction which prevents air from escaping into the esophagus when the stomach is inflated. Bowel occluding clamps, or similar clamping apparatus, are then placed around the efferent limb or limbs of the gastrojejunal anastomosis, and the stomach is inflated with air by means of a syringe or inflation pump injecting air into the rearward end of the primary lumen via the inflation arm. When the air pressure within the stomach reaches a predetermined pressure, air escapes through the pressure regulating valve inside or connected to the second arm at the rearward end of the primary lumen. The pressure relief valve maintains air pressure in the stomach within a predetermined range most suitable for anastomotic testing. The anastomosis is submersed in fluid within the abdominal cavity, and bubbles escaping from around the anastomosis indicate an anastomotic leak, which can then be repaired. Yet other embodiments of the invention are methods for testing for anastomotic leaks whereby a pressure relief valve is arranged in communication with a channel in a rigid or flexible endoscope and air insufflated into the anastomosed organ can escape through the endoscope and the valve only when the air pressure within the organ exceeds a predetermined pressure.

### BRIEF DESCRIPTION OF THE DRAWINGS

The invention, together with further objects and advantages thereof, may best be understood by reference to the following description taken in conjunction with the accompanying drawings, wherein like reference numerals identify like elements, and wherein:

FIG. 1 is a perspective view of a first embodiment of an air introduction device for use in an anastomotic leak tester in accordance with the invention.

FIG. 2 is a cross-sectional view of the embodiment shown in FIG. 1.

FIG. 3 is a perspective view of an inflation pump for use in combination with the air introduction device in accordance with the invention.

FIG. 4 is a perspective view of the first embodiment of the air introduction device in accordance with the invention shown coupled to the inflation pump shown in FIG. 3 to form a first embodiment of an anastomotic leak tester in accordance with the invention.

FIG. 5 is a diagram showing the use of the anastomotic leak tester of FIG. 4 inserted into the anus of a patient for testing of an anastomosis.

FIG. 6 is a perspective view of a second embodiment of an air introduction device for use in anastomotic leak testing in accordance with the invention.

FIG. 7 is a cross-sectional view of the embodiment shown in FIG. 6.

FIG. 8 is a perspective view of the second embodiment of the air introduction device in accordance with the invention shown coupled to the inflation pump shown in FIG. 3 to form a second embodiment of an anastomotic leak tester in accordance with the invention.

FIG. 9 is a perspective view of the anastomotic leak tester of FIG. 8 showing a laparoscope inserted through the endoscopic port.

FIG. 10 is a diagram showing the use of the anastomotic leak tester of FIG. 9 inserted into the anus of a patient for inspection of an anastomosis.

FIG. 11 is a cross-sectional view of a third embodiment of an air introduction device in accordance with the invention for use with an inflation pump to form a third embodiment of an anastomotic leak tester.

FIG. 12 is a cross-sectional view of a fourth embodiment of an air introduction device in accordance with the invention for use with an inflation pump to form a fourth embodiment of an anastomotic leak tester.

FIG. 12A is a cross-sectional view of a fifth embodiment of an air introduction device in accordance with the invention for use with an inflation pump to form a fifth embodiment of an anastomotic leak tester.

FIG. 12B is a rear view of the air introduction device shown in FIG. 12A.

FIG. 12C is a cross-sectional view of a sixth embodiment of an air introduction device in accordance with the invention for use with an inflation pump to form a sixth embodiment of an anastomotic leak tester.

FIG. 12D is a rear view of the air introduction device shown in FIG. 12C.

FIG. 12E is a cross-sectional view of a seventh embodiment of an air introduction device in accordance with the invention for use with an inflation pump to form a seventh embodiment of an anastomotic leak tester.

FIG. 12F is a rear view of the air introduction device shown in FIG. 12E.

FIG. 12G is a side view partially broken away showing a proctoscope (rigid sigmoidoscope) adapted with a pressure relief mechanism in accordance with the invention for insufflating the bowel.

FIG. 12H is a side view of the pressure relief mechanism shown in FIG. 12G.

FIG. 12I is a rear view of the pressure relief mechanism shown in FIG. 12G.

FIG. 13 is a cross-sectional view of an eighth embodiment of an anastomotic leak tester in accordance with the invention.

FIG. 14 is a cross-sectional view of a ninth embodiment of an anastomotic leak tester in accordance with the invention.

FIG. 15 is a cross-sectional view of a tenth embodiment of an anastomotic leak tester in accordance with the invention.

FIG. 16 is a cross-sectional view of an eleventh embodiment of an anastomotic leak tester in accordance with the invention.

FIG. 17 is a cross-sectional view of a twelfth embodiment of an anastomotic leak tester in accordance with the invention.

FIG. 18 is a cross-sectional view of a thirteenth embodiment of an anastomotic leak tester in accordance with the invention.

FIG. 19 is a cross-sectional view of a fourteenth embodiment of an anastomotic leak tester in accordance with the invention.

FIG. 20 is a cross-sectional view of a fifteenth embodiment of an anastomotic leak tester in accordance with the invention.

FIG. 21 is a cross-sectional view of a sixteenth embodiment of an anastomotic leak tester in accordance with the invention.

FIG. 22 is a partial cross-sectional view of a seventeenth embodiment of an anastomotic leak tester in accordance with the invention, useful for anastomoses to the esophagus or stomach.

### DETAILED DESCRIPTION OF THE INVENTION

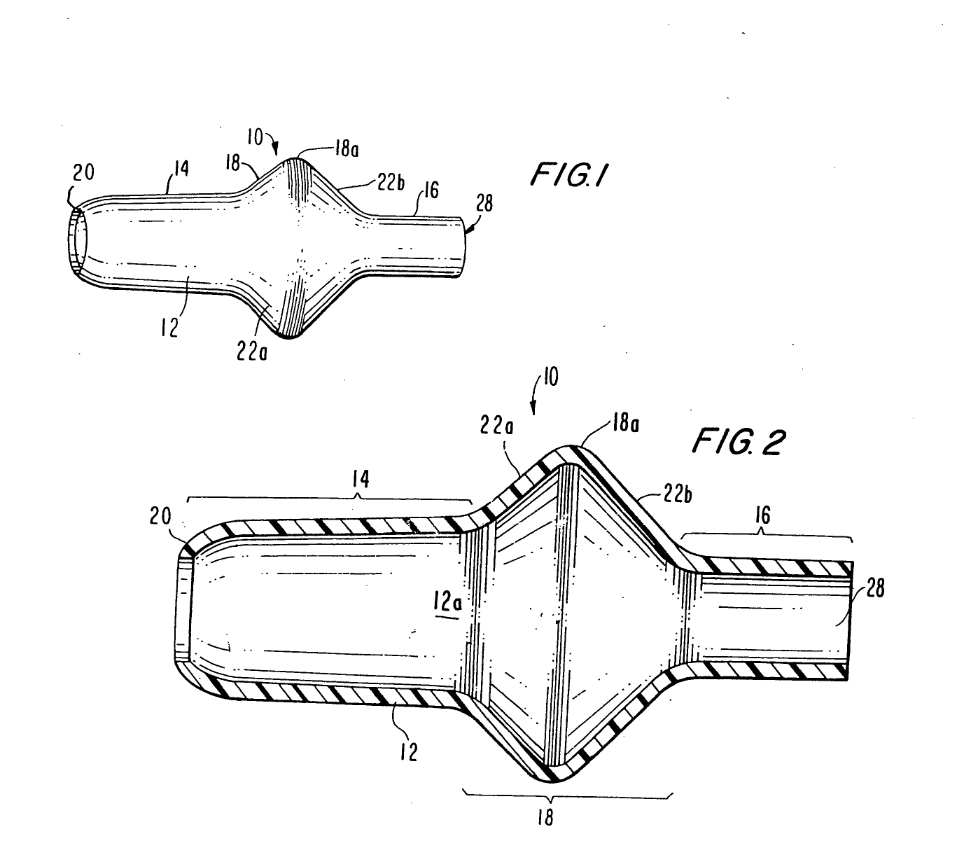

Referring to the accompanying drawings wherein like reference numerals refer to the same or similar elements, FIGS. 1 and 2 show a first embodiment of an air introduction device for anastomotic leak testing in accordance with the invention which is designated generally as 10. The air introduction device 10 comprises a substantially tubular, unitary body 12 defining an interior space 12a and has a proximal segment or portion 14 and a distal segment or portion 16 separated from the proximal portion 14 by an expanded segment or portion 18.

The body 12 of the air introduction device 10 may be made of an elastomer compatible for contact with internal parts of the human body, namely the anal wall and anal opening as discussed below, and fabricated using a dip molding technique to obtain a soft, pliable form. Other plastic manufacturing methods can also be used.

Proximal portion 14 has a substantially uniform inner and outer diameter with the exception that a tip 20 of the proximal portion 14 is rounded or tapered to aid insertion of proximal portion 14 into the anus of an anesthetized patient. The distal portion 16 has a substantially uniform inner and outer diameter smaller than those of the proximal portion 14. Expanded portion 18 has an outer circumferential portion 18a having the largest diameter of the entire body 12 and which is dimensioned to be larger than the opening of most human anuses when they are maximally dilated so that the body 12 cannot be inserted into the anus farther than the outer circumferential portion 18a. That is, the expanded portion 18 serves as insertion-limiting flange which serves to limit the amount of insertion of the proximal portion 14 of the body 12 into the anus. The expanded portion 18 includes two truncated conical surfaces 22a, 22b, one surface 22a tapering from the outer circumferential portion 18a toward the rear of the proximal portion 14 and one surface 22b tapering from the outer circumferential portion 18a toward the front of the distal portion 16.

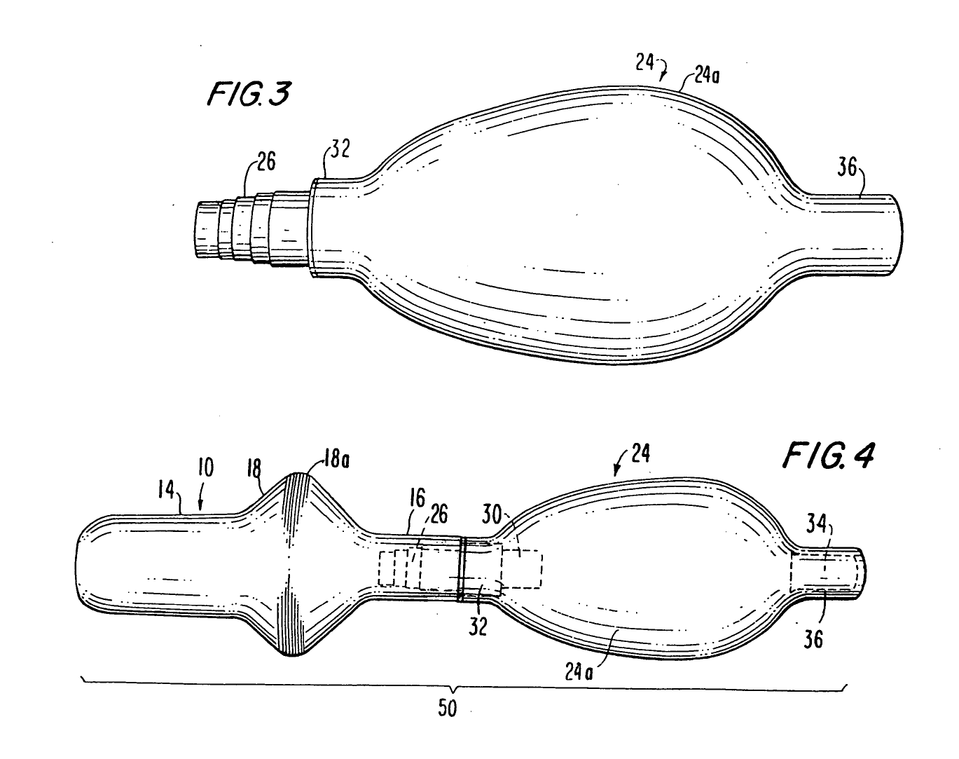

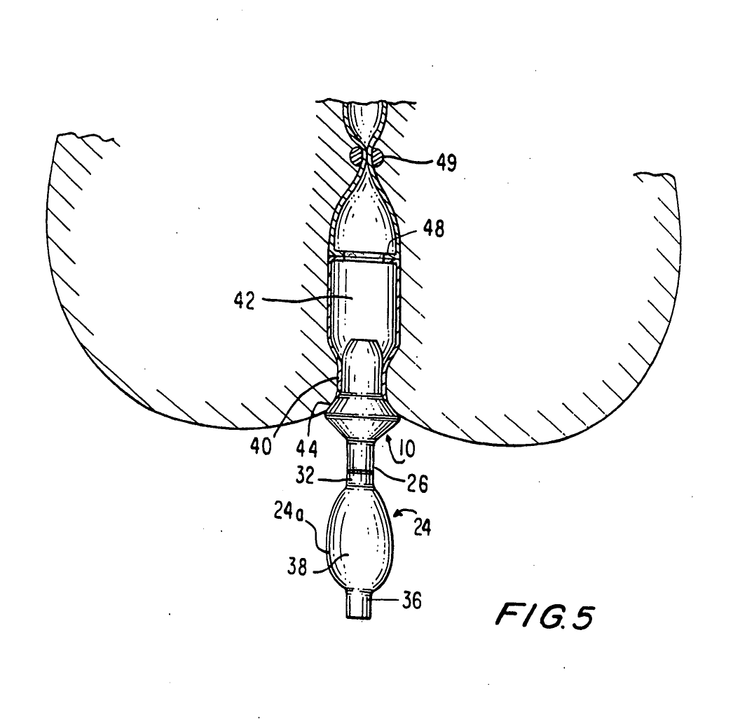

The air introduction device 10 is designed for use with a pumping device such as inflation pump 24 shown in FIG. 3. The inflation pump 24 has a bulbous central portion 24a defining an interior space receivable of air and is coupled to the distal portion 16 of the body 12 (see FIG. 4). More specifically, the inflation pump 24 includes a connector 26 which is insertable into a lumen 28 defined by the distal portion 16 of the air introduction device 10. The inflation pump 24 also includes a valve 30 at a proximal end 32 (housed within the connector 26 of the inflation pump 24 as shown in FIG. 5) and a valve 34 at a distal end 36 (housed within the inflation pump 24). The valves 30,34 permit movement of air in one direction only, specifically in a direction from the distal end 36 to the proximal end 32.

Bulbous central portion 24a of the inflation pump 24 between the valves 30,34 is compressible and when compressed, forces air from a space 38 defined in the interior of the inflation pump 24 through the valve 30 at the proximal end 32 and through the connector 26 (and into the air introduction device 10 when coupled to the inflation pump 24). When the compressing force on the bulbous central portion 24a of the inflation pump 24 is released, air flows into the space 38 through the valve 34 at the distal end 36. Upon the next compression of the bulbous central portion 24a of the inflation pump 24, this air is then directed through the connector 26 into the air introduction device 10. As such, a pumping action is obtained upon repeated compressions and releases of the bulbous central portion 24a of the inflation pump 24.

Referring now to FIG. 5, in one exemplifying use to test whether an anastomosis 48 is leak-proof, the inflation pump 24 is coupled to the air introduction device 10 by inserting connector 26 into the lumen 28 of the distal portion 16 of the air introduction device 10 to thereby form the anastomotic leak tester 50 shown in FIG. 4. The proximal portion 14 of the air introduction device 10 is then inserted into the anus 40 of an anesthetized patient (see FIG. 5). The pelvis is filled with fluid and the bowel above the anastomosis is occluded with a clamp 49. Repeatedly compressing the inflation pump 24 distends the rectum 42 with air. Air cannot escape from the anus 40 because the proximal portion 14 is dimensioned such that its walls dilate the anus 40 whereby the elastic anus constricts around the proximal portion 14 of the air introduction device 10 to form a seal between the walls of the proximal portion 14 and the walls of the anus 40. Thus, air cannot pass between the outer wall of the air introduction device 10 and the wall of the anus 40.

Furthermore, as the rectum 42 is being insufflated by the repeated compression of the inflation pump 24, the air introduction device 10 is pushed inward until the conical surface 22a of the expanded portion 18 presses against the anal verge or opening 44, adding a further seal to prevent the escape of air from the rectum 42. When the rectum 42 is sufficiently distended with air, and no air bubbles are seen in the pelvic fluid, it can be considered that the anastomosis 48 is air tight.

The inflation pump 24 is then detached from the air introduction device 10 while the proximal portion 14 thereof remains in the anus 40, which allows air from the rectum 42 to escape, and allows the bowel to deflate. The air introduction device 10 is then removed out of engagement with the anus 40.

In one exemplifying embodiment, the outer diameter of the walls defining the proximal portion 14 is approximately 1.062 inches, which has been determined to be suitable to allow it to be inserted into the anal canal of an anesthetized patient with ease and at the same time prevent the escape of air from the rectum 42 by passing between the wall of the air introduction device 10 and wall of the anus 40. The largest diameter of the expanded portion 18, i.e., the outer circumferential portion 18a, has an approximate outer diameter of approximately 1.75 inches which is too large to enter the anal canal (without applying excessive force) and which thus abuts and occludes the anal opening 44 when the proximal portion 14 is inserted completely into the anus 40. The expanded portion 18 thus serves to both limit the extent to which the air introduction device 10 is inserted into the rectum 42 and occlude the anal opening 44 to prevent leakage of air around the air introduction device 10. The distal portion 16 has an inner diameter of approximately 0.375 inches, which joins easily with an inexpensive disposable inflation pump 24, which is commercially available, by means of the tubing connector 26 of the inflation pump 24.

These dimensions, while allowing for optimal performance, also permit the air introduction device to be formed by a dip molding process, which is less expensive than other molding processes (e.g. injection molding). If the proximal portion 14 were appreciably thinner, or if the maximum diameter 18 a of the expanded portion 18 were appreciably larger, the mandrel, around which the elastomeric device is molded, could not be readily removed from the device (unless the walls of the device were made thinner, which would render the device too flimsy for use.)

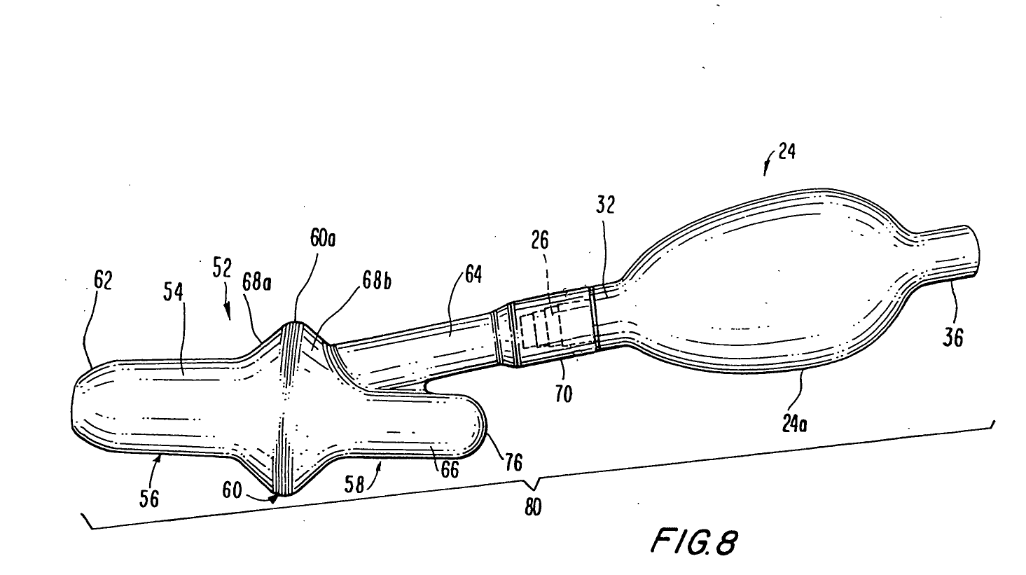

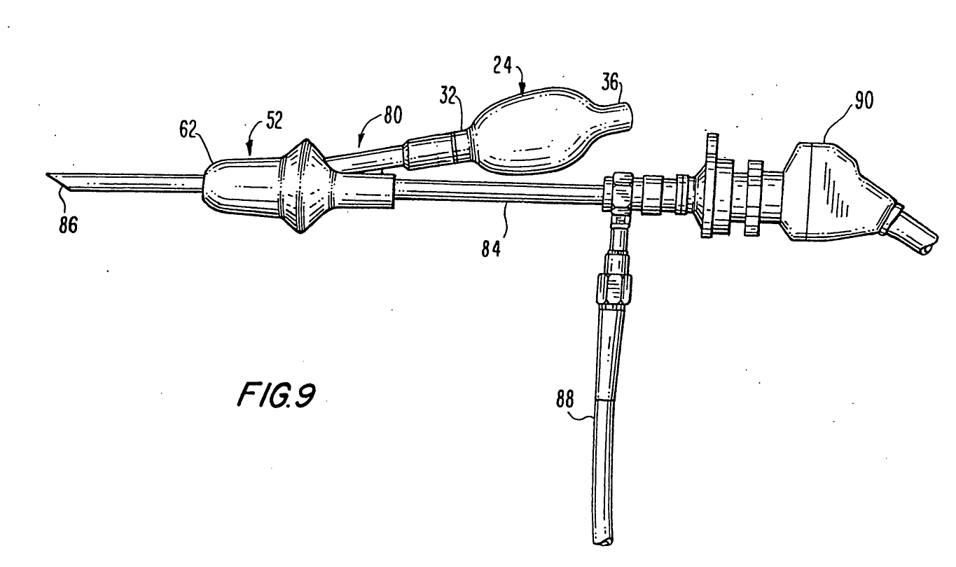

Referring now to FIGS. 6-10, a second embodiment of an air introduction device in accordance with the invention is designated generally as 52 and comprises a unitary body 54 defining an interior space 54a and having a proximal segment or portion 56 and a distal segment or portion 58 separated from the proximal portion 56 by an expanded segment or portion 60. The body 54 of the air introduction device 52 may be made of an elastomer compatible for contact with internal parts of the human body and fabricated using a dip molding technique to obtain a soft, pliable form. Other plastic manufacturing methods can also be used.

The proximal portion 56 has a substantially uniform inner and outer diameter with the exception that a tip 62 is rounded or tapered to aid insertion of proximal portion 56 into the anus of an anesthetized patient. The distal portion 58 has two arms 64,66 extending from the expanded portion 60. Expanded portion 60 has an outer circumferential portion 60a having the largest diameter thereof and which is dimensioned to be larger than the opening of most human anuses when they are maximally dilated so that the air introduction device 52 cannot be inserted into the anus farther than the circumferential portion 60a. The expanded portion 60 includes two truncated conical surfaces 68a, 68b, one surface 68a tapering from the outer circumferential portion 60a toward the rear of the proximal portion 56 and one surface 68b tapering from the outer circumferential portion 60a toward the front of the distal portion 58.

The first arm 64 on the distal portion 58 constitutes an inflation side arm defining an inflation port whereas the second arm 66 constitutes an endoscopic side arm defining an endoscopic port. More specifically, the first arm 64 has a distal end 70 and lumen 72 which is designed to receive the connector 26 of the inflation pump 24 (the same as shown in FIG. 3) and communicates with the interior space 54a of the body 54. The second arm 66 defines a lumen 74 between the interior space 54a and a closed end 76. The lumen 74 is separated from the interior space 54a by a constriction 78, which may be approximately 10 mm in diameter. In the non-limiting illustrated embodiment, a longitudinal axis of the second arm 66 (designated L1) is parallel to a longitudinal axis of the proximal portion 56 and the expanded portion 60 (designated L2), but offset therefrom.

The air introduction device 52 may include the same features as the air introduction device 10 described above, to the extent possible.

In one exemplifying use to test whether an anastomosis 48 is leak-proof, the air introduction device 52 is coupled to the inflation pump 24 by inserting connector 26 of the inflation pump 24 into lumen 72 of the distal portion 58 of the air introduction device 52 to thereby form the anastomotic leak tester 80 shown in FIG. 8. The proximal portion 56 of the air introduction device 52 is then inserted into the anus 40 of an anesthetized patient (see FIG. 10). The pelvis is filled with fluid and the bowel above the anastomosis is occluded with clamp 49. The inflation pump 24 is compressed several times, distending the rectum 42 with air. As explained above, air cannot escape from the anus 40 because of the seals formed by the proximal portion 56 of the air introduction device 52 against the anal wall and the expanded portion 60 of the air introduction device 52 against the anal opening 44. When the rectum 42 is sufficiently distended with air, and no air bubbles are seen in the pelvic fluid, it is can be considered that the anastomosis 48 is air tight and leak-proof. The air introduction device 52 is then removed from the anus.

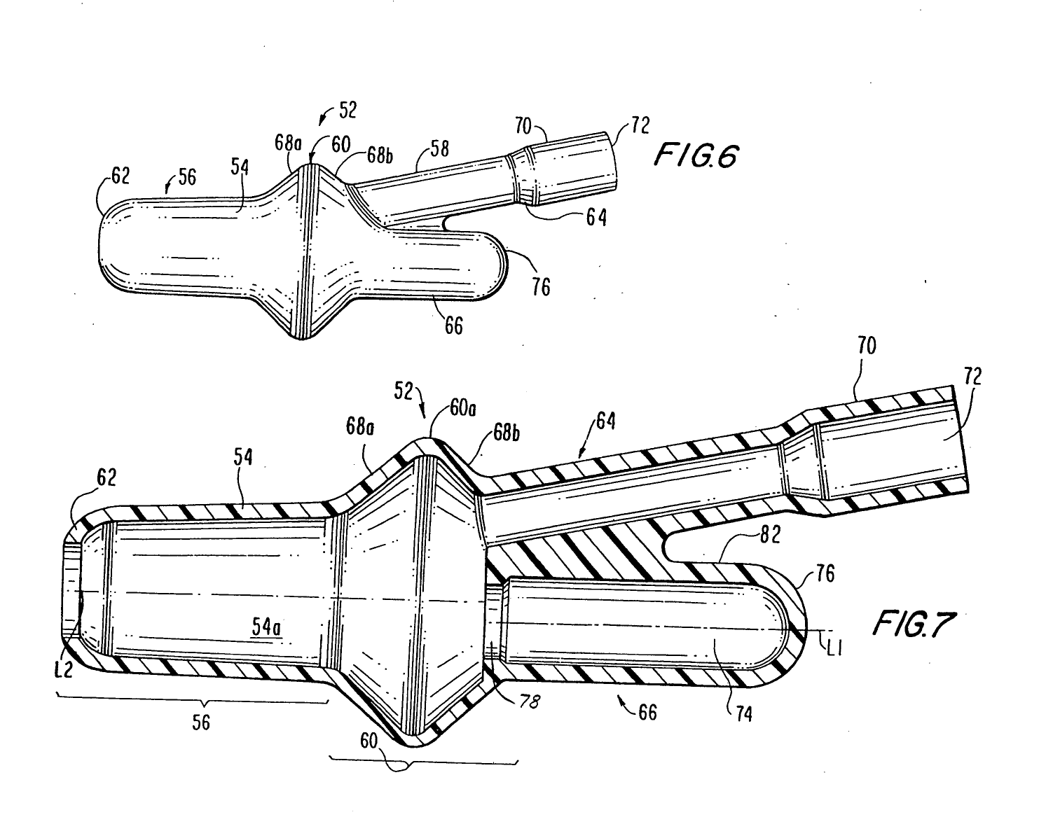

A second way to evaluate the anastomosis involves a visual inspection of the internal aspect of the anastomosis 48. To this end, the closed end 76 of the second arm 66 is severed off, for example, with a scissors at point 82 (FIG. 7) between the closed end 76 and a location where the second arm 66 joins the first arm 64.

A laparoscope 84 is then inserted into lumen 74 so that the tip or proximal end 86 of the laparoscope 84 sits within space 54a of the body 54. The laparoscope 84 is attached to a fiberoptic cable 88 which is connected to a light source (not shown). Laparoscope 84 may be attached to a camera 90 which is connected to an image processor (not shown) and video display (not shown). The proximal portion 56 of the air introduction device 52 (attached to the inflation pump 24) is then inserted into the anus 40 so that the expanded portion 60 abuts against the anal verge 44. The inflation pump 24 is compressed several times, distending the rectum 42 with air. The tip 86 of the laparoscope 84 is then advanced from the position in the space 54a in the elastomeric body 54 into the rectum 42 while viewing the advancing progress directly through the lense of the laparoscope or on a video display to a point that permits a clear picture of the anastomosis 48 (see FIG. 10).

The constriction 78 serves to provide a seal against the laparoscope 84 to prevent air and fluid in the interior space 54a from being released from the body 54 via the lumen 74.

A laparoscope with an angled tip may be used, and the laparoscope may be rotated to obtain an excellent view of the inner aspect of the bowel anastomosis 48 on the video display. Bleeding from the anastomosis 48 can be easily identified and anastomotic defects visualized. Corrective surgical action can then be taken.

When the visual inspection is complete, the laparoscope 84 is removed with the leak tester 80 remaining in place. The bowel is deflated and then the leak tester 80 removed out of engagement with the patient.

Embodiment 52 can alternatively be provided with end 76 being open instead of closed, that is arm 66 already having been severed at point 82. Leak testing with air can still be performed, if the opening is occluded by a finger, and this embodiment is immediately available for use with the laparoscope 84 without the surgeon having to severe arm 66.

Referring now to FIG. 11, a third embodiment of an air introduction device in accordance with the invention is designated generally as 92 and comprises a unitary body 94 defining an interior space 94a and having a proximal segment or portion 96 and a distal segment or portion 98 separated from the proximal portion 96 by an expanded segment or portion 100. The body 94 of the air introduction device 92 may be made of an elastomer compatible for contact with internal parts of the human body and fabricated using a dip molding technique to obtain a soft, pliable form. Other plastic manufacturing methods can also be used.

The proximal portion 96 has a substantially uniform inner and outer diameter with the exception that a tip 102 is rounded or tapered to aid insertion of proximal portion 96 into the anus of an anesthetized patient. The distal portion 98 has two arms 104, 106 extending from the expanded portion 100. Expanded portion 100 has an outer circumferential portion 100a having the largest diameter thereof and which is dimensioned to be larger than the opening of most human anuses when they are maximally dilated so that the air introduction device 92 cannot be inserted into the anus farther than the circumferential portion 100a. The expanded portion 100 includes two truncated conical surfaces 108a, 108b, one surface 108a tapering from the outer circumferential portion 100a toward the rear of the proximal portion 96 and one surface 108b tapering from the outer circumferential portion 100a toward the front of the distal portion 98.

The first arm 104 on the distal portion 98 constitutes an inflation side arm defining an inflation port whereas the second arm 106 includes a pressure relief valve 110 arranged in a lumen 118. The first arm 104 has a distal end 112 and a lumen 114 which is designed to receive the connector 26 of the inflation pump 24 (the same as shown in FIG. 3) and communicates with the interior space 94a of the body 94.

Valve 110 is a pressure relief valve which allows air to escape from the bowel when a specific air pressure within the rectum 42 is reached. This prevents over-inflation of and damage to the anastomosis. A bill-shaped extension 115 is attached to a distal end 116 of the second arm 106 and may be separately formed from an elastic material such as rubber and fixed to the distal end 116 of the second arm 106 by an adhesive or other comparable attachment mechanism.

When air escapes the bowel through the second arm 106, i.e., through valve 110, it passes through extension 115 causing upper and/or lower flaps 117 of the extension 115 to vibrate. This produces an audible signal which alerts the surgeon to the fact that the desired air pressure within the bowel has been reached. Other mechanisms for causing an audible (e.g. a whistle) or visual indication when air is released via pressure relief valve 110 can be used instead of the flaps 117.

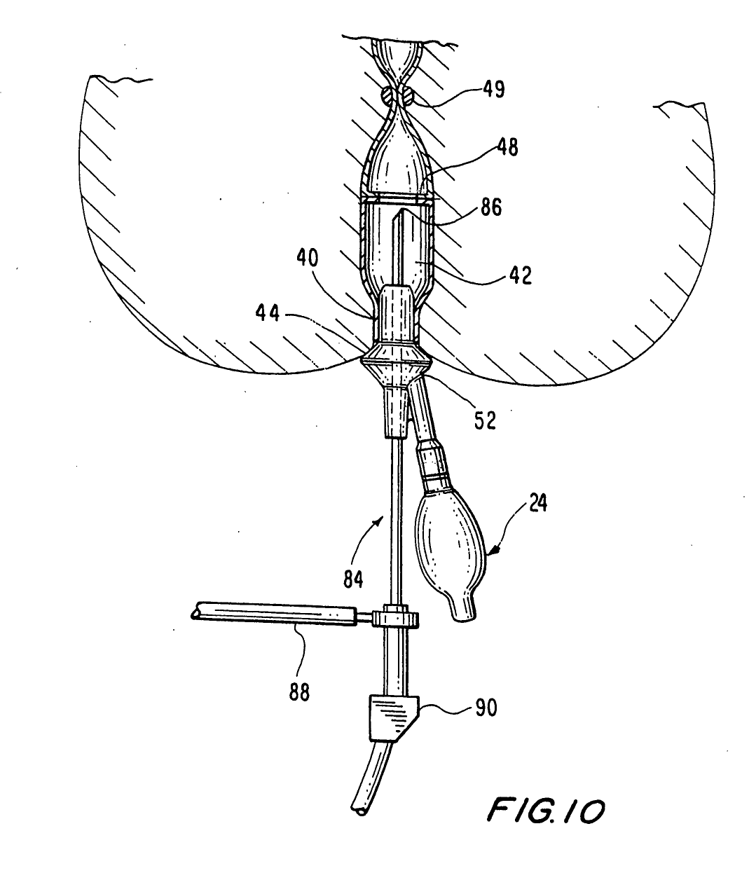

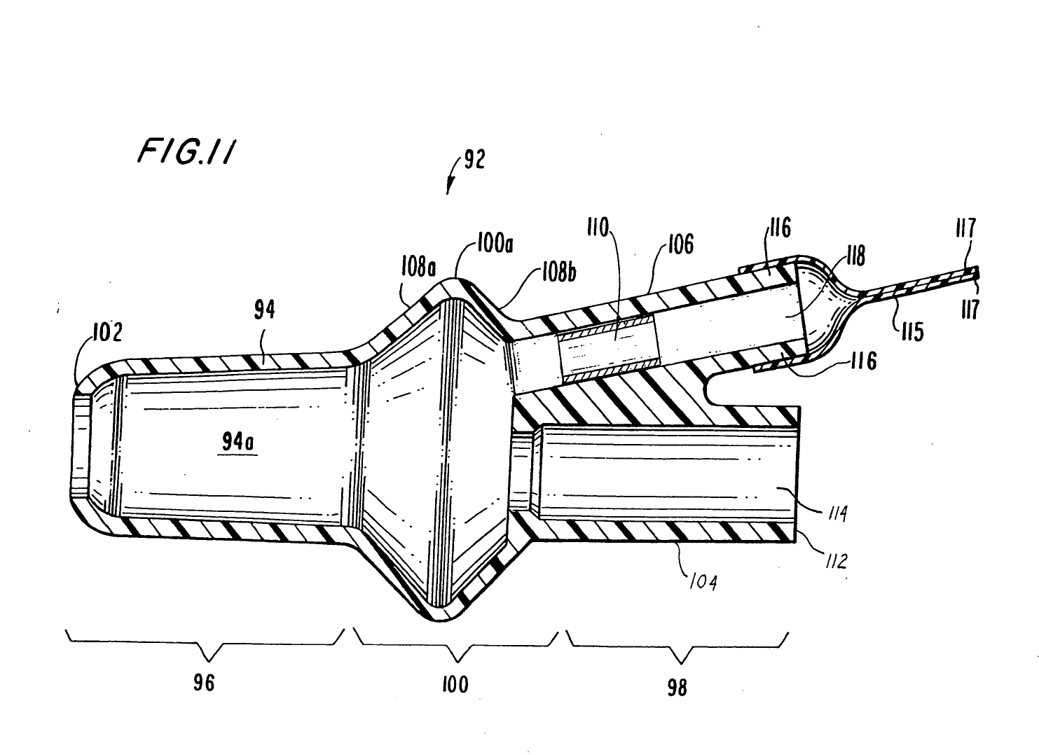

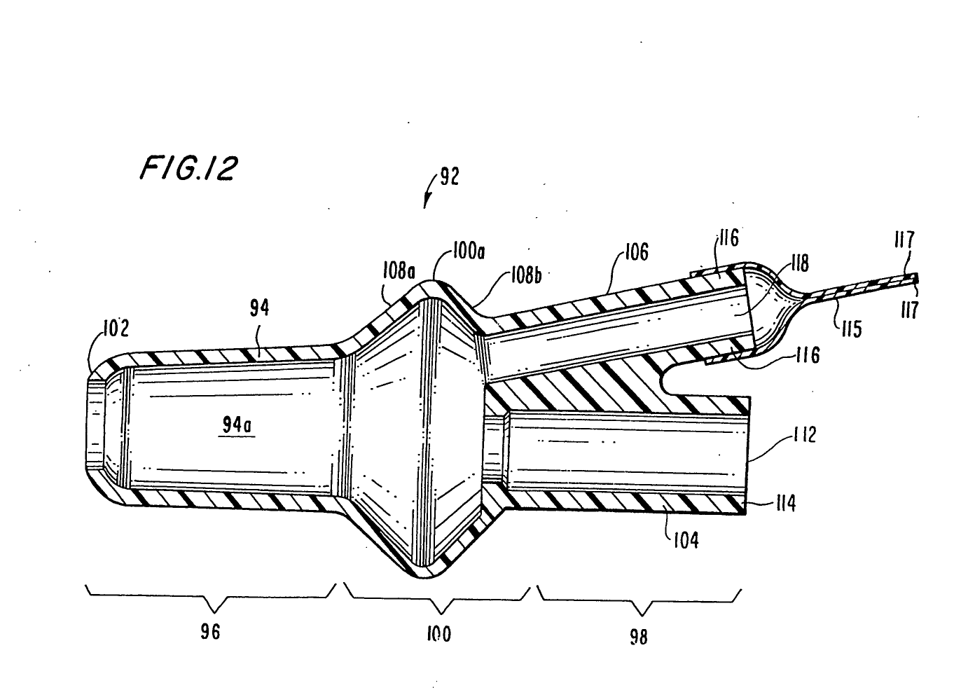

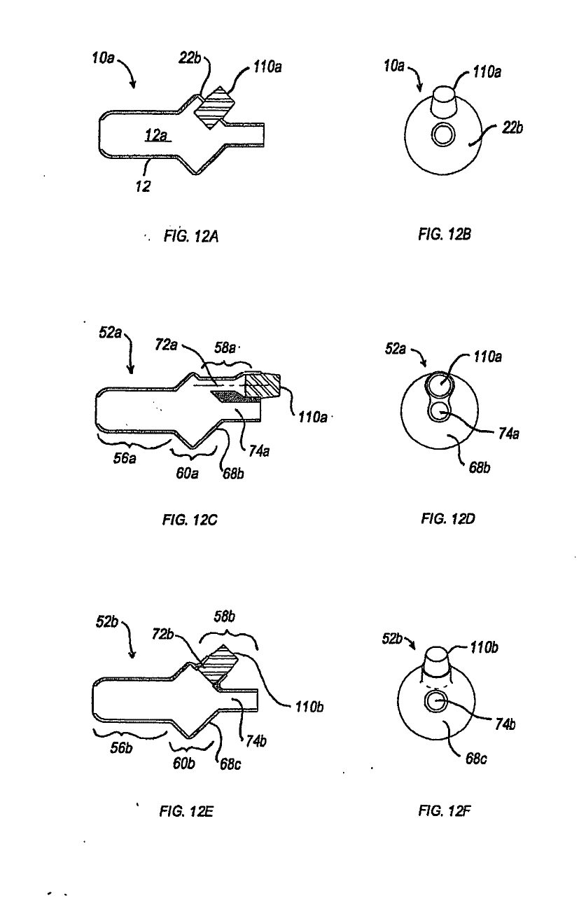

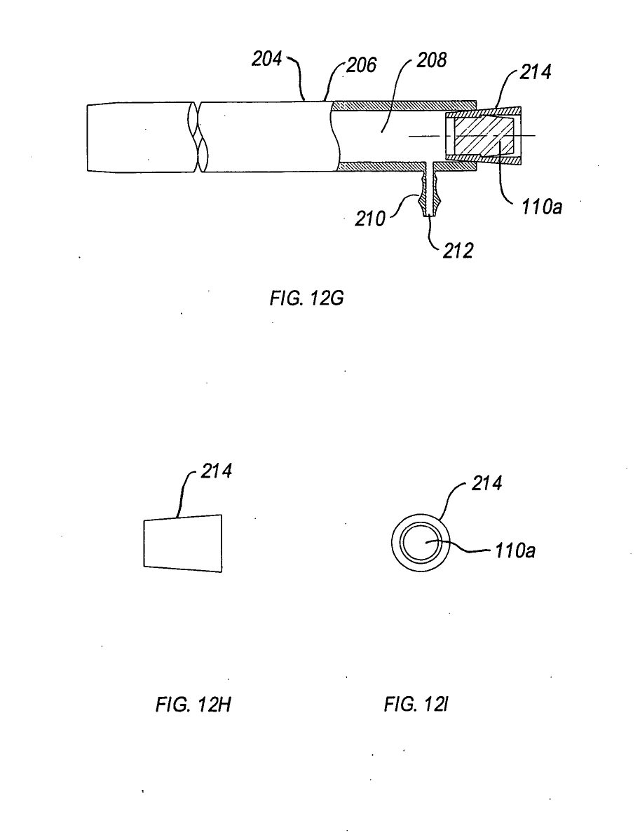

Alternatively, the extension 115 may be designed to permit passage of air between the flaps 117 only when a specific air pressure is reached in the bowel. That is, the extension 115 may function both as a pressure release valve and a mechanism to produce an audible signal. In this case, a separate pressure relief valve 110 is not necessary and not provided, as shown in FIG. 12.

Other indications of pressure may be used, for example, a low cost inflatable manometer such as those manufactured by Dielectrics, Inc., or such as the Mercury Medical Disposable Pressure Manometer sold by Keomed Inc., and can be arranged in connection with the air introduction device so that the air pressure in the bowel can be read off the manometer's scale. Such a pressure indicating mechanism can be employed in any of the embodiments disclosed herein, with or without a pressure relief valve.

The air introduction device 92 may include the same features as the air introduction devices 10 and 52 described above, to the extent possible.

Referring now to FIGS. 12A-12F, additional constructions of air introduction devices are shown which regulate air pressure within the bowel and/or generate or provide an audible indication or audible signal when the desired air pressure within the bowel has been reached. The desired air pressure is that air pressure at which the bowel is sufficiently distended to allow for proper testing of the anastomosis yet without over-distending the bowel. It has been found that the desired air pressure should ideally be between about 25 cm water and about 30 cm water.

In the embodiment shown in FIGS. 12A and 12B, the air introduction device 10a is similar to air introduction device 10 with the major exception that a pressure relief valve 110a is arranged to extend through the surface 22b of the expanded portion 18. For example, the pressure relief valve 110a can be pushed or otherwise inserted into an aperture formed in surface 22b so that one portion is inside the body 12 and one portion is exterior of the body 12. Generally, the pressure relief valve 110a can be positioned in connection with the body 12 at any position in which its inlet is in flow communication with the interior space 12a of the body 12, which interior space 12a would be at the same pressure as the air within the bowel during use of the air introduction device 10 to distend the bowel. The outlet of the pressure relief valve 110a may be positioned anywhere in flow communication with the ambient atmosphere, which can either be a location within the body 12 leading to the ambient atmosphere or entirely exterior of the body 12. When the pressure in the bowel exceeds the design pressure threshold of the pressure relief valve 110a (that pressure which causes the pressure relief valve 1110a to open up and form a conduit for air from the interior of the body 12 to the ambient atmosphere), air enters the pressure relief valve 110a through the inlet in communication with the interior space 12a and leaves the pressure relief valve 110a through the outlet exterior of the body 12.

Pressure relief valve 110a preferably has a construction which generates or causes an audible indication when air flows therethrough, which corresponds to an indication that the pressure in the bowel has reached the design pressure threshold of the pressure relief valve 110a, the audible indication being of sufficient strength and intensity to be heard by a surgeon operating on the patient. Specifically, pressure relief valve 110a can be medical grade pressure relief valve Part#: 730ROA5G manufactured by Halkey-Roberts which produces a vibratory sound, similar to a fart, when the pressure in the bowel reaches or exceeds about 30 cm water, which is an optimal pressure for testing for anastomotic leaks. Once the pressure within the bowel exceeds the cracking (opening) pressure of such a pressure relief valve 110a it will not be possible to further distend the bowel (any additional air inflow would be equaled by a corresponding air outflow through the pressure relief valve 110a). Thus, this pressure relief valve 110a may serve three important purposes, i.e., it acts as a pressure relief valve to limit the maximum pressure to which the bowel can be inflated, it acts as a pressure regulatory valve by maintaining pressure in the bowel lumen within a range optimal for anastomotic leak testing and it provides an audible signal when the air pressure desired for anastomotic leak testing of the bowel is reached.

Alternatively, the pressure regulatory valve may be designed to whistle when air passes through it. This may give the surgeon a clearer audible signal that the desired testing pressure has been reached.

The air introduction device 10a may include the same features as the air introduction device 10 described above, to the extent possible.

In the embodiment shown in FIGS. 12C and 12D, air introduction device 52a has a proximal portion 56a and expanded portion 60a substantially similar to air introduction device 52 but with a significantly different distal portion 58a than distal portion 58 in air introduction device 52. Distal portion 58a of air introduction device 52a comprises two lumens, a pressure relief lumen 72a and an inflation lumen 74a. Inflation lumen 74a is approximately centered relative to surface 68b. Pressure relief valve 110a is arranged partially or completely within lumen 72a (see FIG. 12C) and functions in the same manner as described above. A passage is formed in lumen 72a between the interior of the expanded portion 60a and the inlet of the pressure relief valve 110a, this passage being substantially parallel to the inflation lumen 74a (although other angular orientation are possible).

The air introduction device 52a may include the same features as the air introduction device 52 described above, to the extent possible.

In the embodiment shown in FIGS. 12E and 12F, air introduction device 52b has a proximal portion 56b and expanded portion 60b substantially similar to air introduction devices 52 and 52a, but with a significantly different distal portion 58b than distal portion 58 in air introduction device 52 and distal portion 58a in air introduction device 52a. Distal portion 58b of air introduction device 52b comprises two lumens, a pressure relief lumen 72b and an inflation lumen 74b. Inflation lumen 74b is approximately centered relative to surface 68c. Pressure relief valve 110b is arranged partially within lumen 72b (see FIG. 12E) and functions in the same manner as described above. Lumen 72b extends from the expanded portion 60b with little or no passage between the interior of the expanded portion 60b and the inlet of the pressure relief valve 110b, i.e., the inlet of the pressure relief valve 110b is proximate the interior of the expanded portion 60b (compare FIG. 12E to FIG. 12C). Also, lumen 72b can be arranged to extend substantially perpendicular to the rear-facing surface of the expanded portion 60b.

The air introduction device 52b may include the same features as the air introduction devices 52 and 52a described above, to the extent possible.

In another embodiment of the invention, the distal portion of the body may include three arms, an inflation port on one arm (for mating with an inflation pump), an endoscopic port on another arm (for enabling passage of an endoscope therethrough) and a third arm containing a pressure relief valve and signal producing component.

In the embodiments of the anastomotic leak testers described above, the inflation pump 24 includes the connector 26 with the valve 30 therein. However, in other embodiments, instead of the connector, the inflation pump 24 may be provided with a tubular passage downstream of the valve 30 in the air flow direction and a connector arranged in connection with the air introduction device and adapted to enter into the tubular passage of the inflation pump (as shown in FIG. 21 below).

Referring now to FIGS. 12G-121, pressure relief valve 110a can be used in connection with an endoscope such as a proctoscope (rigid sigmoidoscope) 204, which is an alternative instrument to insufflate the bowel instead of the anastomotic leak testers described herein including an inflation pump and an air introduction device. Proctoscope 204 includes an elongate portion 206 having a channel 208 therein, and an air inflow arm 210 extending outward from the elongate portion 206 and defining a lumen 212 in flow communication with the channel 208. In use, a tube leading from an inflation pump, is attached to the air inflow arm 210 and activated to cause air flow through channel 208 into the bowel once the forward end of the proctoscope has been inserted into the bowel through the anus.

To enable its use with proctoscope 204, pressure relief valve 110a is placed into a stopper 214 made of, e.g., rubber or silicone, and inserted into the distal end of channel 208. As such, when the pressure in the bowel reaches the design pressure threshold of pressure relief valve 110a, i.e., a pressure anywhere in the range of about 25 cm water to about 30 cm water, air will flow through pressure relief valve 110a, pressure within the bowel will be maintained in the desired range, and the air flow through the valve 110a may generate an audible signal. Instead of an audible signal via pressure relief valve 110a, other audible or visual signals can be generated when air flows through pressure relief valve 110a, for example, as described above with respect to FIGS. 11 and 12A-12F.

Alternatively, a pressure relief valve may be arranged in connection with an instrument channel of a flexible sigmoidoscope, so that when air pressure within an organ exceeds a predetermined pressure, air may escape the organ through the instrument channel and pressure relief valve to thereby regulate air pressure within the organ. Additionally, the air flow through the valve may generate an audible signal.

FIGS. 13-21 show additional embodiments of an anastomotic leak tester in accordance with the invention which can be used essentially in the same manner as described above to test an anastomosis.

FIG. 13 shows an embodiment wherein an anastomotic leak tester 120 includes inflation pump 24 and an air introduction device having a tubular body 122 with a proximal end 124 which is closed. The body 122 is made primarily of a flexible, elastomeric material and has a connector portion 126 at a distal end and an aperture 128 on a side close to the proximal end. In use, the body 122 of the air introduction device is placed such that the aperture 128 is inside the patient so that upon compression of the bulbous, compressible central portion 24a of the inflation pump 24, air flows into the body 122 and out of the aperture 128 into the patient's bowel.

FIG. 14 shows an anastomotic leak tester 130 which includes inflation pump 24 and an air introduction device having a tubular body 132 which is similar to body 122 except that the aperture 134 is formed at the tip of the proximal end 124, i.e., it is an axial aperture. As shown, the proximal end 124 of the body 132 has a curved form with the aperture 134 in the center.

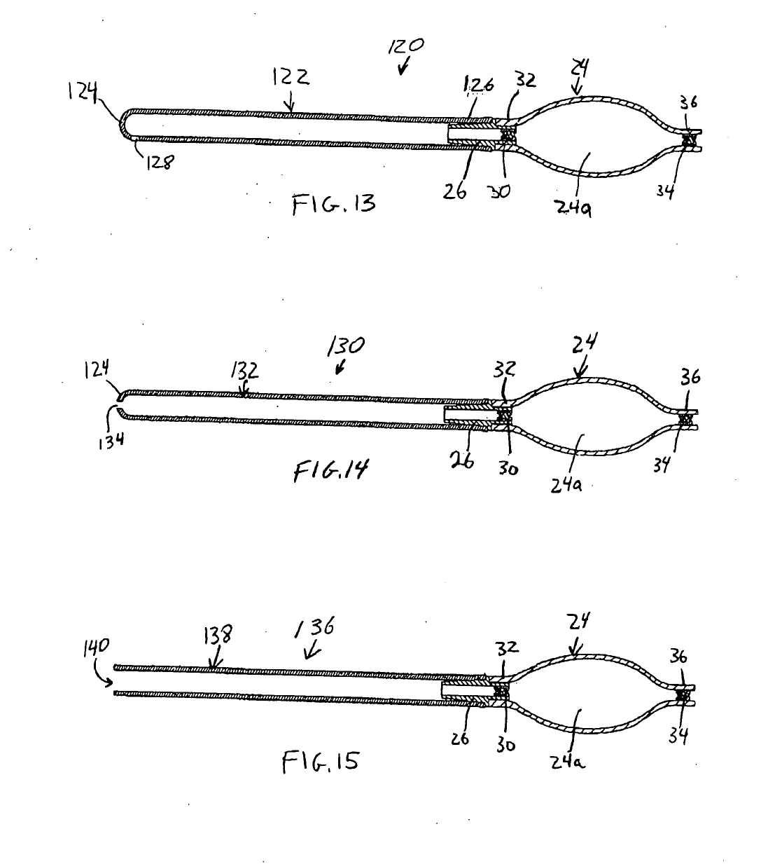

FIG. 15 shows an anastomotic leak tester 136 which includes inflation pump 24 and an air introduction device having a tubular body 138 which is similar to body 132 except that the aperture 140 extends over the entire cross-section thereof, i.e., the diameter of the inner passage defined by the tubular body 138 is substantially constant at least adjacent the proximal end and possibly along its entire length.

FIG. 16 shows an anastomotic leak tester 142 including an inflation pump and air introduction device integrally formed with one another. Leak tester 142 includes a bulbous compressible portion 144 defining an interior space receivable of air, an inflow valve 146 at a distal end 148, an outflow valve 150 and a tubular portion 152 with a proximal end 154 which is closed. At least the tubular portion 152 is made of a flexible, elastomeric material and has an aperture 156 on a side close to the proximal end 154. The valves 146, 150 permit movement of air in one direction only, specifically in a direction from the distal end 148 to the proximal end 154. The use of anastomotic leak tester 142 is essentially the same as for the anastomotic leak testers described above.

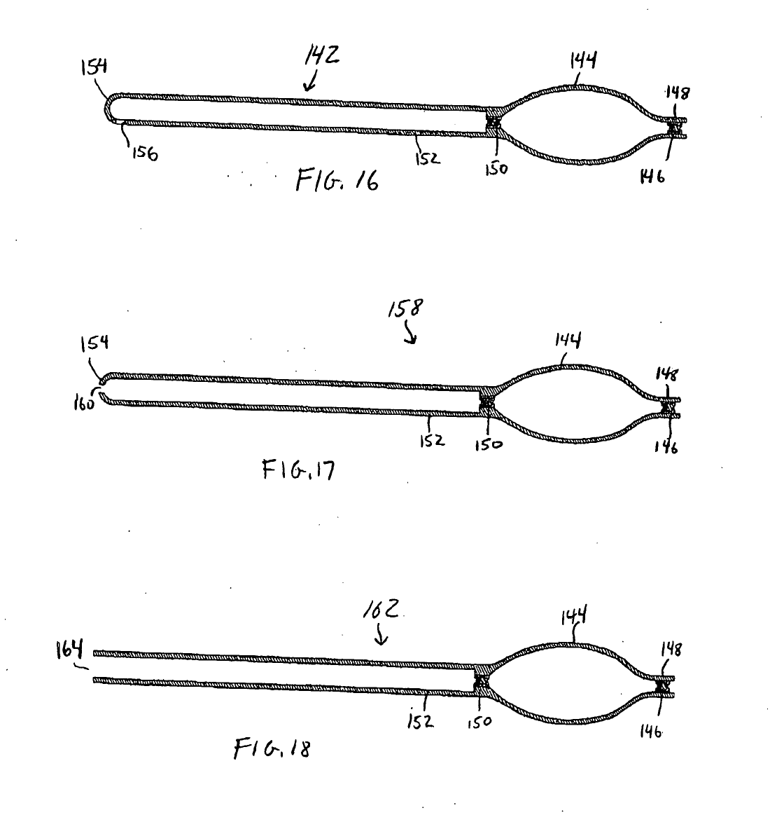

FIG. 17 shows an anastomotic leak tester 158 which is similar to anastomotic leak tester 142 except that the aperture 160 is formed at the tip of the proximal end 154, i.e., an axial aperture. Thus, the proximal end 154 has a curved form with the aperture 160 in the center.

FIG. 18 shows an anastomotic leak tester 162 which is similar to anastomotic leak tester 158 except that the aperture 164 extends over the entire cross-section of the tubular portion 152, i.e., the diameter of the inner passage defined by the tubular portion 152 is substantially constant at least adjacent the proximal end and possibly along its entire length.

In the embodiments shown in FIGS. 16-18, the outflow valve 150 is arranged adjacent the compressible portion 144. Alternatively, the outflow valve can be arranged at the proximal end 154 of the tubular portion 152 immediately before or close to the aperture 156, 160.

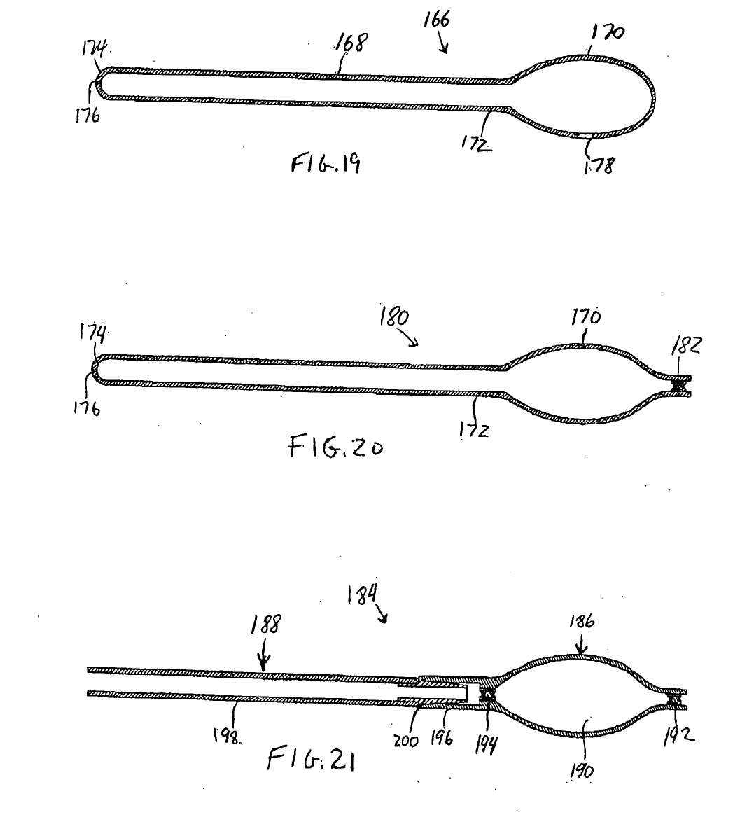

FIG. 19 shows an anastomotic leak tester 166 including a body 168 which provides the functions of both an inflation pump and an air introduction device. Body 168 is made of a flexible, elastomeric material and has a bulbous compressible portion 170 at a distal end defining an interior space receivable of air and a tubular portion 172 with a proximal end 174 having a slit 176 (the air introduction portion). An aperture 178 is formed in the bulbous compressible portion 170. To provide air flow through the body 168, the surgeon covers the aperture 178, e.g., with a finger, and compresses compressible portion 170. This forces the slit 176 to open with air flowing from the body 168 into the patient's bowel. The slit 176 spontaneously closes when air flow stops, that is, when compressible portion 170 is compressed no further. The surgeon then removes his finger, opening aperture 178, causing air to flow through the aperture 178 into the compressible portion 170. The compressible portion 170 expands as it fills with air, and the insufflation process is repeated until the bowel is appropriately distended.

FIG. 20 shows an anastomotic leak tester 180 that is similar to anastomotic leak tester 166 except that instead of aperture 178 in the bulbous compressible portion 170, the air flow into the compressible portion 170 is provided by a valve 182 arranged at the distal end of the body 168 to provide a one-way flow of air into a compressible portion 170 of the body 168.

FIG. 21 shows an anastomotic leak tester 184 including an inflation pump 186 and an air introduction device 188 which is separable from the inflation pump 186. The inflation pump 186 has a compressible central portion 190 defining an interior space receivable of air and includes an inflow valve 192 at a distal end and an outflow valve 194 at a proximal end. Inflation pump 186 also includes a substantially tubular, connector portion 196 communicating with the outflow valve 194. The valves 192, 194 permit movement of air in one direction only, specifically in a direction from the distal end to the proximal end. Air introduction device 188 comprises a tube 198 and a connector 200 which is arranged to removably fit in the connector portion 196 of the inflation pump 186. Compression of the compressible portion 190 causes air flow through the valve 194 and through the tube 198 into the patient's bowel. Instead of having an open end as shown, the tube 198 can also be provided with one or more apertures, e.g., an aperture on the side at the proximal end as shown in FIG. 13 or an axial aperture as shown in FIG. 14, or valve 194 may be eliminated and the proximal end of tube 198 provided with a slit as shown in FIGS. 19 and 20.

In the embodiments described above wherein the inflation pump is separable from the air introduction device and two valves are used to provide a uni-directional flow of air, instead of arranging the outflow valve in connection with the inflation pump, it is also possible to arrange the outflow valve in connection with the air introduction device. The connector can be arranged on either the same component as the outflow valve or on the other component.

Also, in the embodiments described above with reference to FIGS. 13-21, it is also possible to provide the air introduction device or tubular portion of the anastomotic leak tester with the expanded portion described above and shown in, e.g., FIGS. 1 and 2, as well as other features thereof.

Moreover, the air introduction device, when separate from the inflation pump, and the anastomotic leak tester, when including a tubular portion and inflation pump portion, can be dip molded to form a single use, disposable product which is inexpensive to manufacture. In addition, the air introduction device or tubular portion of the anastomotic leak tester can be made entirely or only partially of a transparent or translucent material so that it can be more readily ascertained when blood is present in the bowel (indicative of a problem with the surgery) since the blood will be seen on and/or in the air introduction device or tubular portion.

Furthermore, the proximal tip of the air introduction device, when separate from the inflation pump, or proximal tip of the tubular portion of the anastomotic leak tester, when integral with inflation pump, may include a bulbous calibration tip.

When an anastomotic leak tester described above has a separate air introduction device and inflation pump, the inflation pump connects to an axial end of the air introduction device. By contrast, in prior art proctoscopes (rigid sigmoidoscopes), an inflation pump is connected to a port on a side of the proctoscope and the lens gate is situated at the axial end of the proctoscope. An axial connection simplifies and improves the connection and sealing between the inflation pump and the air introduction device, and allows for more facile manipulation of the device by the surgeon.

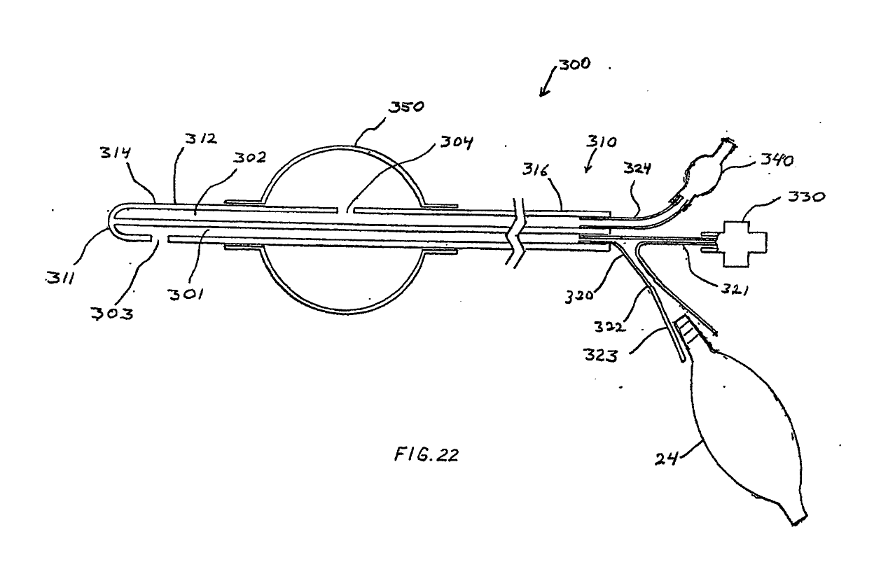

Referring now to FIG. 22, another embodiment of an anastomotic leak tester 300 in accordance with the invention is useful for testing anastomoses to the stomach or esophagus. Anastomotic leak tester 300 includes an air introduction device 310 and an inflation pump 24. Air introduction device 310 comprises a dual lumen insertion tube 312 of sufficient length to enable the forward end 314 to be situated in close proximity to the gastric or esophageal anastomosis while the rearward end 316 remains external to the nose or mouth when the insertion tube 312 is inserted through the nose or mouth of a patient. This is a dual lumen tube, which is comprised of an elastomeric material compatible with the human body, such as polyvinyl chloride or silicone, and has a gastrointestinal inflation lumen 301 and an inflatable cuff inflation lumen 302. Insertion tube 312 has an aperture 303 in forward end 314 at or near forward tip 311 so that gastrointestinal inflation lumen 301 is in flow communication with the interior of the anastomosed organ in which forward end 314 lies. At the rearward end 316 of insertion tube 312, one limb or arm 321 of a Y-shaped connector 320 connects a pressure relief valve 330 to insertion tube 312 so that the interior of valve 330 is in flow communication with gastrointestinal inflation lumen 301 and the interior of the anastomosed organ through aperture 303. A second limb or arm 322 of Y-connector 320 ends in a connector 323 to which inflation pump 24, or syringe, or other type of inflation device, is attachable.

Insertion tube 312 has a second aperture 304 communicating with cuff inflation lumen 302. Overlying aperture 304, an inflatable cuff 350 is fixed. Rearward end 316 of insertion tube 312 is connected to a cuff inflation valve 340 by means of a connector tube 324. A syringe is adaptable to the cuff inflation valve 340 so that air can be injected through valve 340, connector tube 324, cuff inflation lumen 302 and aperture 304 to inflate cuff 350. Cuff inflation valve 340 prevents deflation of the cuff 350 unless a syringe is actively reapplied to the valve 340 to withdraw air. Instead of cuff inflation valve 340, it is possible to use a clamp which closes a tube leading to the cuff inflation lumen 302.

In use, the forward end 314 of insertion tube 312 is inserted through the nose or mouth, through the pharynx into the distal esophagus or stomach. If the anastomosis to be tested is to the stomach, the inflatable cuff 350 is positioned in the stomach and then inflated, and then the tube 312 is pulled rearward so that the cuff 350 abuts against the stomach wall near or at the gastroesophageal junction. Fully inflated, the cuff 350 is too large to be withdrawn into the esophagus, and provides a seal at the gastroesophageal junction which prevents air from escaping from the stomach into the esophagus when the stomach is inflated. Occluding clamps, or similar instruments, are then placed around the efferent limb or limbs of the gastrointestinal anastomosis, and the stomach is inflated with air by means of a syringe or inflation pump 24 by injecting air through connector tube 320, gastrointestinal inflation lumen 301 and aperture 303. When the air pressure within the stomach reaches a predetermined pressure, air escapes through the pressure relief valve 330.

Pressure relief valve 330 maintains air pressure in the stomach within a predetermined range most suitable for anastomotic testing. This range is readily known to or otherwise determinable by one skilled in the art. The anastomosis is submersed in fluid within the abdominal cavity, and bubbles escaping from the anastomosis indicate an anastomotic leak, which should then be repaired and the test repeated.

Alternatively, if the proximal or entire stomach has been surgically removed and an anastomosis formed between the intestines and the esophagus, distal end 314 of insertion tube 312 is placed in the esophagus in proximity to the anastomosis. The inflatable cuff 350 is inflated in the esophagus to form a seal against the esophageal wall. The efferent limbs of bowel are occluded with bowel occluding clamps, the distal esophagus and anastomosed bowel are inflated by the means described above and submersed in a pool of fluid that is checked for bubbles indicating a leak. If a leak is present it is repaired and the test is repeated.

While particular embodiments of the invention have been shown and described, it will be obvious to those skilled in the art that changes and modifications may be made without departing from the invention in its broader aspects, and, therefore, the aim in the appended claims is to cover all such changes and modifications as fall within the true spirit and scope of the invention.

We claim:

1. An anastomotic leak tester for testing for leaks in a gastrointestinal anastomosis in a patient, comprising:
an air introduction system adapted to be inserted into the patient's gastrointestinal tract through a natural body orifice of the patient and enabling air flow therethrough into the gastrointestinal tract; and
regulating means arranged in connection with said air introduction system for regulating air pressure in the patient's gastrointestinal tract.

2. The anastomotic leak tester of claim 1, wherein said regulating means are arranged to release air from the patient's gastrointestinal tract when the air pressure in the patient's gastrointestinal tract exceeds a predetermined pressure.

3. The anastomotic leak tester of claim 1, wherein said regulating means comprise a pressure relief valve having an inlet communicating with an interior space of said air introduction system communicating with the patient's gastrointestinal tract and arranged to allow air to be released from the patient's gastrointestinal tract when air pressure in the patient's gastrointestinal tract exceeds a predetermined pressure.

4. The anastomotic leak tester of claim 3, wherein said pressure relief valve generates an audible indication when air pressure in the patient's gastrointestinal tract exceeds the predetermined pressure.

5. The anastomotic leak tester of claim 1, wherein said air introduction system comprises an air introduction device including a body having an interior space.

6. The anastomotic leak tester of claim 5, wherein said regulating means comprise a pressure relief valve arranged to extend through a surface of said body such that an inlet of said pressure relief valve communicates with said interior space of said body and an outlet of said pressure relief valve communicates with the ambient atmosphere.

7. The anastomotic leak tester of claim 5, wherein said air introduction system is adapted to be inserted into the patient's bowel through the patient's anus and said body includes an expanded portion for limiting insertion of said body into the anus, said regulating means comprising a pressure relief valve arranged to extend through a rear-facing surface of said expanded portion of said body such that an inlet of said pressure relief valve communicates with said interior space of said body and an outlet of said pressure relief valve communicates with the ambient atmosphere.

8. The anastomotic leak tester of claim 5, wherein said air introduction system is adapted to be inserted into the patient's bowel through the patient's anus and said body includes an expanded portion for limiting insertion of said body into the anus, and a distal portion comprising two lumens each of which communicates with said expanded portion, a first one of said lumens being connectable to an inflation pump, said regulating means comprising a pressure relief valve arranged at least partially inside a second one of said lumens such that an inlet of said pressure relief valve communicates with said interior space of said body and an outlet of said pressure relief valve communicates with the ambient atmosphere.

9. The anastomotic leak tester of claim 8, wherein said pressure relief valve is arranged partially in said second lumen such that an inlet of said pressure relief valve is proximate an interior of said expanded portion.

10. The anastomotic leak tester of claim 1, wherein said regulating means comprise a pressure relief valve having a design pressure threshold equal to a predetermined desired air pressure in the patient's gastrointestinal tract such that when the air pressure in the gastrointestinal tract is above the predetermined air pressure, said pressure relief valve opens a conduit for air flow from the gastrointestinal tract to the ambient atmosphere.

11. The anastomotic leak tester of claim 1, wherein said regulating means comprise a pressure relief valve arranged to release pressure from the patient's gastrointestinal tract and to generate a sound when air flows therethrough.

12. The anastomotic leak tester of claim 1, wherein said regulating means comprise a pressure relief valve arranged to release pressure from the patient's gastrointestinal tract and generate an audible signal when the air pressure in the patient's gastrointestinal tract exceeds about 30 cm water.

13. The anastomotic leak tester of claim 1, wherein said air introduction system comprises an endoscope defining a channel and an air inflow port, and said regulating means comprise a pressure relief valve arranged to release pressure from the patient's gastrointestinal tract when air pressure in the patient's gastrointestinal tract exceeds the predetermined air pressure.

14. The anastomotic leak tester of claim 13, wherein said endoscope is a sigmoidoscope and further comprising a stopper arranged at a rearwardl end of said channel, said pressure relief valve being contained within said stopper.

15. The anastomotic leak tester of claim 1, wherein said air introduction system comprises:
an insertion tube having a forward end adapted to be positioned proximate the anastomosis and a rearward end adapted to remain external to the patient, said insertion tube having a gastrointestinal inflation lumen, an inflatable cuff inflation lumen, a first aperture communicating with said cuff inflation lumen, an inflatable cuff overlying said first aperture and a second aperture communicating with said gastrointestinal inflation lumen and arranged forward of said inflation cuff,
said regulating means comprising a pressure relief valve communicating with said gastrointestinal inflation lumen.

16. The anastomotic leak tester of claim 15, further comprising a Y-connector engaging with said gastrointestinal inflation lumen at said rearward end of said insertion tube, said pressure relief valve being arranged in a first arm of said Y-connector and a second arm of said Y-connector being connectable to an inflation pump.

17. An anastomotic leak tester for testing for leaks in a gastrointestinal anastomosis in a patient, comprising:
an air introduction system adapted to be inserted into the patient's gastrointestinal tract and enabling air flow therethrough into the gastrointestinal tract; and
signal indication means arranged in connection with said air introduction system for generating a signal when a specific air pressure in the patient's gastrointestinal tract is attained.

18. The anastomotic leak tester of claim 17, wherein said signal indicating means comprise a flow path having an inlet communicating with an interior space of said air introduction system communicating with the patient's gastrointestinal tract and arranged to generate an audible signal when air flows therethrough as a result of pressure in the patient's gastrointestinal tract exceeding a predetermined pressure.

19. A method for distending a portion of a gastrointestinal tract for anastomotic leak testing, comprising:
inserting an air introduction system into the patient's gastrointestinal tract through a patient's natural body orifice;
directing air into the patient's gastrointestinal tract through the air introduction system; and
generating a signal when air pressure in the patient's gastrointestinal tract exceeds a predetermined air pressure.

20. The method of claim 19, wherein the signal generated is an audible signal.

21. The method of claim 19, wherein the signal generated is a visual signal.

22. A method for distending a portion of a gastrointestinal tract for anastomotic leak testing, comprising:
inserting an air introduction system into the patient's gastrointestinal tract through a patient's natural body orifice;
directing air into the patient's gastrointestinal tract through the air introduction system; and
automatically regulating air pressure in the patient's gastrointestinal tract to cause release of air from the patient's gastrointestinal tract when the air pressure in the patient's gastrointestinal tract exceeds a predetermined pressure.

23. The method of claim 22, when the body orifice is the patient's mouth.

24. The method of claim 22, when the body orifice is the patient's nose.

25. The method of claim 22, when the body orifice is the patient's anus.

26. The method of claim 22, wherein the step of regulating the air pressure in the patient's gastrointestinal tract comprises arranging a pressure relief valve in connection with the air introduction system in a position in which it has an inlet communicating with an interior space of the air introduction system communicating with the patient's gastrointestinal tract and an outlet communicating with the ambient atmosphere.

27. The method of claim 26, further comprising constructing the pressure relief valve to generate an audible indication when air pressure in the patient's gastrointestinal tract exceeds the predetermined pressure.

28. The method of claim 22, wherein the air introduction system comprises an air introduction device including a body having an interior space, the step of regulating the air pressure in the patient's gastrointestinal tract comprising arranging a pressure relief valve to extend through a surface of the body such that an inlet of the pressure relief valve communicates with the interior space of said body and an outlet of the pressure relief valve communicates with the ambient atmosphere.

29. The method of claim 22, wherein the step of regulating the air pressure in the patient's gastrointestinal tract comprises arranging a pressure relief valve having a design pressure threshold equal to the predetermined air pressure in connection with the air introduction system such that when the air pressure in the gastrointestinal tract is above the predetermined air pressure, the pressure relief valve opens a conduit for air flow from the gastrointestinal tract to the ambient atmosphere.

30. The method of claim 22, where the predetermined pressure is 30 cm of water.

31. The method of claim 22, wherein the air introduction system comprises an endoscope defining a channel and an air inflow port, the step of automatically regulating the air pressure in the patient's gastrointestinal tract comprising arranging a pressure relief valve in communication with the interior of the endoscope, the pressure relief valve releasing pressure from the patient's gastrointestinal tract when air pressure in the patient's gastrointestinal tract exceeds the predetermined air pressure.

32. The method of claim 31, wherein the endoscope is a sigmoidoscope and further comprising arranging the pressure relief valve in a stopper and inserting the stopper into a distal end of the sigmoidoscope channel.

33. The method of claim 22, wherein the air introduction system includes an insertion tube having a forward end adapted to be positioned proximate the anastomosis and a rearward end adapted to remain external to the patient, the insertion tube having a gastrointestinal inflation lumen, an inflatable cuff inflation lumen, and an inflatable cuff communicating with said inflatable cuff inflation lumen, the method further comprising:
inflating the inflatable cuff via the inflatable cuff inflation lumen to occlude the gastrointestinal tract after the forward end of the insertion tube is positioned proximate the anastomosis, and
distending the anastomosis with air delivered through the gastrointestinal inflation lumen,
the step of automatically regulating air pressure in the patient's gastrointestinal tract comprising arranging a pressure relief valve in communication with the gastrointestinal inflation lumen.

34. A method for gastrointestinal anastomotic leak testing, comprising:
filling a body cavity of a patient with fluid so as to submerse the anastomosis with that fluid;
occluding the gastrointestinal tract proximal and distal to the anastomosis; then
distending the anastomosis according to the method of claim 22 after the gastrointestinal tract is occluded; and
checking for anastomotic leaks based on the presence of air bubbles in the pelvic fluid.
